SEQUENCE LISTING (5 sequences)

SEQ ID NO: 1 - protein, 127 aa, Camelus bactrianus
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 2 - protein, 274 aa, Artificial Sequence
  Antigen binding domain
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 3 - protein, 605 aa, Artificial Sequence
  Chimeric antigen receptor
MLLLVTSLLLCELPHPAFLLIPMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITEFGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 752 aa, Artificial Sequence
  Chimeric antigen receptor
MLLLVTSLLLCELPHPAFLLIPMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITEFGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 20 aa, Artificial Sequence
  Linking peptide
GGGGSGGGGSGGGGSGGGGS

(19) United States
(12) Patent Application Publication
(10) Pub. No.: US 2023/0159636 A1
(43) Pub. Date: May 25, 2023

(54) CD7 CHIMERIC ANTIGEN RECEPTOR-MODIFIED NK-92MI CELL AND USE THEREOF

(71) Applicant: PERSONGEN BIOTHERAPEUTICS (SUZHOU) CO., LTD., Suzhou, Jiangsu (CN)

(72) Inventors: Lin YANG, Suzhou, Jiangsu (CN); Fengtao YOU, Suzhou, Jiangsu (CN)

(21) Appl. No.: 17/419,420

(22) PCT Filed: Dec. 30, 2019
(86) PCT No.: PCT/CN2019/129928

§ 371 Date: Dec. 15, 2021

(30) Foreign Application Priority Data
Dec. 29, 2018 (CN) 201811641769.6

(51) Int. Cl. C07K 16/28; A61K 35/17; A61P 35/02; C07K 14/705; C07K 14/725

(52) U.S. Cl. CPC C07K 16/2803; A61K 35/17; A61P 35/02; C07K 14/70521; C07K 14/70578; C07K 14/7051; A61K 2039/505

(57) ABSTRACT

The present invention provides a CD7 chimeric antigen receptor-modified NK-92MI cell and use thereof. In particular, the present invention provides an engineered NK cell expressing a chimeric antigen receptor (CAR), said CAR having an antigen-binding domain containing a nanobody VHH sequence targeting CD73. Said NK cell of the present invention can effectively kill tumor cells, especially T cell tumors, and has a good therapeutic effect on T cell leukemia (such as T-ALL).

### TECHNICAL FIELD

The present disclosure belongs to the field of biomedicine, in particular to NK-92MI cells modified by CD7 chimeric antigen receptors and uses thereof.

### BACKGROUND

T-cell malignancies represent a type of blood system cancer, with high recurrence and mortality rates in children and adults. There is currently no effective or targeted therapy. T-cell acute lymphoblastic leukemia (T-ALL) is a highly heterogeneous hematological malignancy, accounting for 25% of adult acute lymphoblastic leukemia cases and 15% of pediatric acute lymphoblastic leukemia cases. Currently, T-ALL treatment strategies include intensive chemotherapy, allogeneic hematopoietic stem cell transplantation (allo-HSCT), antiviral therapy and molecular targeted therapy. However, intensive chemotherapy and allo-HSCT usually cannot prevent and treat refractory relapses. For those patients who relapse after initial treatment, the remission rate of salvage chemotherapy is about 20-40%. Although hematopoietic stem cell transplantation is the only curable option, there is a risk of death.

In recent years, chimeric antigen receptor T cell (CAR T) therapy has shown very effective therapeutic effects as a powerful new adoptive immunotherapy technique for a variety of solid and hematological cancers, most notably for the treatment of B-cell lymphocytic leukemia and lymphoma. CAR-T therapy uses modified patient T lymphocytes to target and eliminate malignant tumors in a major histocompatibility complex-independent manner. The key to the effective application of this technology is to select a suitable CAR target. The best target antigen should be expressed only in tumor cells, not in normal cells, or on cells that have a clinical response plan after the normal cells are temporarily missing. Therefore, B cell-derived leukemia and lymphoma can be treated with CAR targeting CD19 or CD22, because CD19 and CD22 are only expressed by B lymphoid cells. Infusion of autologous T cells expressing anti-CD19-CAR into patients with refractory B-cell leukemia and lymphoma can lead to significant clinical responses. These results provide undisputed evidence to support the huge potential of this technology in clinical applications.

However, the design of CARs for T cell tumors still faces great challenges. This is because the same antigens will be expressed on normal T cells and T cell malignant tumors, causing CAR-T cells to kill each other.

Therefore, there is an urgent need to develop drugs and methods that can effectively treat T-cell tumors in this field.

### SUMMARY OF THE INVENTION

The purpose of the present disclosure is to provide a drug and method that can effectively treat T cell tumors.

Another purpose of the present disclosure is to provide NK-92MI cells modified by CD7 chimeric antigen receptors and uses thereof.

In a first aspect of the present invention, it provides an engineered NK cell, which expresses a chimeric antigen receptor CAR, and the antigen binding domain of the CAR contains VHH sequences of nanobody targeting CD7.

In another preferred embodiment, the antigen binding domain contains n VHH sequences of nanobody targeting CD7, wherein n is a positive integer of 1-5, preferably n is a positive integer of 1-3, and more preferably n is 1 or 2.

In another preferred embodiment, when n≥2, the antigen binding domain also contains a linking peptide La between each VHH sequences of nanobody targeting CD7.

In another preferred embodiment, the linking peptide La has a length of 5-25, preferably 10-20 amino acids.

In another preferred embodiment, the structure of the antigen binding domain is V_{HH} or V_{HH}-I-V_{HH}, wherein the V_{HH} is the VHH sequence of nanobody targeting CD7, and I is none or a linking peptide La.

In another preferred embodiment, the antigen binding domain is one or two VHH sequences of nanobody targeting CD7.

In another preferred embodiment, the VHH sequence of the nanobody targeting CD7 is shown in SEQ ID NO.: 1.

In another preferred embodiment, the sequence of V_{HH}-I-V_{HH} is shown in SEQ ID NO.: 2.

In another preferred embodiment, the sequence of the linking peptide La is shown in SEQ ID NO.: 5.

In another preferred embodiment, the antigen binding domain comprises a sequence as shown in SEQ ID NO.: 1 or 2.

In another preferred embodiment, the sequence of the antigen binding domain is as shown in SEQ ID NO.: 1 or 2.

In another preferred embodiment, the sequence of the antigen binding domain is of at least 70%, preferably at least 75%, 80%, 85%, 90%, more preferably at least 95%, 96%, 97%, 98% or more than 99% sequence identity to the sequence as shown in SEQ ID NO.: 1 or 2.

In another preferred embodiment, the antigen binding domain targets or binds to human CD7.

In another preferred embodiment, the structure of the CAR is as shown in Formula I:

L-S-H-TM-C-CD3ζ (I)

wherein, each “-” is independently a linking peptide or peptide bond;

L is an optional signal peptide sequence;

S is an antigen binding domain;

H is an optional hinge region;

TM is a transmembrane domain;

C is a co-stimulatory signal molecule;

CD3ζ is a cytoplasmic signal transduction sequence derived from CD3ζ.

In another preferred embodiment, the structure of the CAR is L-V_{HH}-H-TM-C-CD3ζ or L-V_{HH}-La-V_{HH}-H-TM-C-CD3ζ, wherein each element is defined as above and La is a linking peptide.

In another preferred embodiment, the L is a signal peptide of a protein selected from the group consisting of CD8, CD28, GM-CSF, and a combination thereof.

In another preferred embodiment, the L is a signal peptide derived from GM-CSF.

In another preferred embodiment, the sequence of the L is as shown in positions 1-22 of SEQ ID NO.: 3.

In another preferred embodiment, the H is a hinge region of a protein selected from the group consisting of CD8, CD28, CD137, Fc, and a combination thereof.

In another preferred embodiment, the H is a hinge region derived from Fc.

In another preferred embodiment, the sequence of the H is as shown in positions 154-382 of SEQ ID NO.: 3.

In another preferred embodiment, the TM is a transmembrane region of a protein selected from the group consisting of CD8, CD28, CD137, and a combination thereof.

In another preferred embodiment, the TM is a transmembrane region derived from CD28.

In another preferred embodiment, the sequence of the TM is as shown in positions 383-407 of SEQ ID NO.: 3.

In another preferred embodiment, the C is a co-stimulatory signal molecule of a protein selected from the group consisting of CD28, CD137 (4-1BB), ICOS (CD278), and a combination thereof.

In another preferred embodiment, the C comprises co-stimulatory signal molecules derived from CD28 and/or 4-1BB.

In another preferred embodiment, the C consists of a CD28-derived co-stimulatory signal molecule and a 4-1BB-derived co-stimulatory signal molecule.

In another preferred embodiment, the sequence of the C is as shown in positions 408-493 of SEQ ID NO.: 3.

In another preferred embodiment, the CAR has an amino acid sequence as shown in SEQ ID NO.: 3 or 4.

In another preferred embodiment, the NK cells are ex vivo.

In another preferred embodiment, the NK cells are autologous or allogeneic.

In another preferred embodiment, the NK cells are human or non-human mammalian cells, preferably human cells.

In another preferred embodiment, the NK cells are NK92 cells, preferably NK92MI cells.

In a second aspect of the present invention, it provides a chimeric antigen receptor CAR, and the antigen binding domain of the CAR contains a VHH sequence of nanobody targeting CD7.

In another preferred embodiment, the antigen binding domain contains n VHH sequences of nanobody targeting CD7, where n is a positive integer of 1-5, preferably n is a positive integer of 1-3, and more preferably n is 1 or 2.

In another preferred embodiment, the structure of the antigen binding domain is VHH or V_{HH}-I-V_{HH}, wherein the V_{HH} is the VHH sequence of nanobody targeting CD7, and I is none or a linking peptide La.

In another preferred embodiment, the antigen binding domain is one or two VHH sequences of nanobody targeting CD7.

In another preferred embodiment, the VHH sequence of the nanobody targeting CD7 is shown in SEQ ID NO.: 1.

In another preferred embodiment, the sequence of V_{HH}-I-V_{HH} is shown in SEQ ID NO.: 2.

In another preferred embodiment, the sequence of the antigen binding domain is as shown in SEQ ID NO.: 1 or 2.

In another preferred embodiment, the antigen binding domain targets or binds to human CD7.

In another preferred embodiment, the structure of the CAR is as shown in Formula I:

L-S-H-TM-C-CD3ζ (I)

wherein, each “-” is independently a linking peptide or peptide bond;

The definitions of L, S, H, TM, C and CD3ζ are described above.

In another preferred embodiment, the structure of the CAR is L-V_{HH}-H-TM-C-CD3ζ or L-V_{HH}-La-V_{HH}-H-TM-C-CD3ζ, wherein each element is defined as described above.

In another preferred embodiment, the CAR has an amino acid sequence as shown in SEQ ID NO.: 3 or 4.

In a third aspect of the present invention, it provides a nucleic acid molecule encoding the chimeric antigen receptor CAR as described in the second aspect of the present invention.

In a fourth aspect of the present invention, it provides a vector comprising a nucleic acid molecule as described in the third aspect of the present invention.

In another preferred embodiment, the vector comprises DNA, RNA.

In another preferred embodiment, the vector is selected from the group consisting of a plasmid, a viral vector, a transposon, and a combination thereof.

In another preferred embodiment, the vector includes a DNA virus, a retrovirus vector.

In another preferred embodiment, the vector is selected from the group consisting of a lentivirus vector, an adenovirus vector, an adeno-associated virus vector, and a combination thereof.

In another preferred embodiment, the vector is a lentivirus vector.

The present invention also provides a host cell, which expresses the CAR according to the second aspect of the present invention; and/or

the genome of the host cell is integrated with an exogenous nucleic acid molecule according to the third aspect of the present invention; and/or

the host cell contains the vector according to the fourth aspect of the present invention.

In another preferred embodiment, the cell is an isolated cell, and/or the cell is a genetically engineered cell.

In another preferred embodiment, the host cell is a human or non-human mammalian cell, preferably a human immune cell.

In another preferred embodiment, the host cell is a NK cell or a T cell.

In a fifth aspect of the present invention, it provides a formulation which comprises the engineered NK cells of the first aspect of the present invention, or the nucleic acid molecules of the third aspect of the present invention, or the vector of the fourth aspect of the present invention, and a pharmaceutically acceptable carrier, diluent or excipient.

In another preferred embodiment, the formulation is a liquid formulation.

In another preferred embodiment, the dosage form of the formulation comprises an injection.

In another preferred embodiment, the concentration of the engineered NK cells in the formulation is 1×10³-1×10⁸ cells/ml, preferably 1×10⁴-1×10⁷ cells/ml.

In a sixth aspect of the present invention, it provides the use of the engineered NK cells of the first aspect of the present invention, the chimeric antigen receptor CAR of the second aspect of the present invention, the nucleic acid molecules of the third aspect of the present invention, or the vectors of the fourth aspect of the present invention for the preparation of drugs or preparations for the prevention and/or treatment of cancer or tumors.

In another preferred embodiment, the tumor is selected from the group consisting of hematological tumors, solid tumors, and a combination thereof.

In another preferred embodiment, the hematological tumor is selected from the group consisting of acute myeloid leukemia (AML), multiple myeloma (MM), chronic lymphocytic leukemia (CLL), acute lymphoblastic leukemia (ALL), diffuse large B-cell lymphoma (DLBCL), and a combination thereof.

In another preferred embodiment, the hematological tumor is T-cell acute lymphoblastic leukemia (T-ALL).

In a seventh aspect of the present invention, it provides a method for preparing the engineered NK cell according to the first aspect of the present invention, which comprises the steps of introducing a nucleic acid molecule according to the third aspect of the present invention or a vector according to the fourth aspect of the present invention into a NK cell, thereby obtaining the engineered NK cell.

In an eighth aspect of the present invention, it provides a method of treating disease, which comprises administering to the subject in need the appropriate amount of the engineered NK cells according to the first aspect of the present invention, or the preparation according to the fifth aspect of the present invention.

It should be understood that within the scope of the present invention, the various technical features of the present invention above and the various technical features specifically described hereinafter (as in the embodiments) may be combined with each other to constitute a new or preferred technical solution. Due to space limitations, it is not repeated here.

### DESCRIPTION OF THE DRAWINGS

FIG. 1 shows that the construction of CD7-CAR-NK-92MI and dCD7-CAR-NK-92MI and expression detection thereof. (A) A schematic diagram of a CD7-specific CAR vector. CD7-CAR vector contains signal peptide sequence, monovalent nanobody VHH6 sequence (bivalent nanobody VHH6 sequence), hinge domain (Fc), two costimulatory domains (CD28 and 4-1BB), and the intracellular signal transduction domain CD3ζ. (B) Expression of Fc on the cell surface of NK-92MI, CD7-CAR-NK-92MI and dCD7-CAR-NK-92MI. (C) Detection of CD7-CAR expression by Western blot. The lysates of NK-92MI (lane 1), CD7-NK-92MI (lane 2) and dCD7-NK-92MI (lane 3) cells were isolated by SDS-PAGE under denaturation conditions. Western blot analysis was performed with CD3ζ chain specific mAb, and then detected by HRP-coupled antibody. Locations of the endogenous (16 KD) and chimeric CD3ζfusion proteins (68 KD, 83 KD) are shown in figure.

FIG. 2 shows the expression level of CD7 in NK-92MI cells and T-ALL tumor cells. (A) Changes of CD7 expression in NK-92MI cells after transfected with CD7-CAR. (B) CD7 expression levels in T-ALL tumor cell lines (CCRF-CEM and Jurkat) or Raji cells (CD7 negative target cell lines). (C) Expression of CD7 in primary T-ALL tumor cells (Sample 1).

FIG. 3 shows that CD7-CAR-NK-92MI and dCD7-CAR-NK-92MI cells specifically kill T-ALL cell lines and primary tumor cells expressing CD7 in vitro. (A) CD7-positive T-ALL cell line CCRF-CEM is targeted and lysed by CD7-CARNK-92MI and dCD7-CAR-NK-92MI at the condition of an effector-target ratio of 1:1. (B) CD7-positive Jurkat cells are specifically lysed by CD7-CAR-NK-92MI and dCD7-CAR-NK-92MI cells at the condition of an effector-target ratio of 1:1 or 5:1, 7-AAD negative cell population represents the percentage of remaining Jurkat cells. (C) The cytotoxicity of CD7-CAR-NK-92MI and dCD7-CAR-NK-92MI cells to primary T-ALL tumor cells at the condition of an effector-target ratio of 1:1 or 5:1.

FIG. 4 shows a comparison of the cytotoxicity of different monovalent CD7-CAR-NK92-MI and bivalent dCD7-CAR-NK-92MI monoclonal cell lines to CCRF-CEM cells. (A) The CAR positive rates of 8 monovalent CD7-CAR-NK-92MI cell lines screened were detected. (B) Cytotoxicity of eight monovalent CD7-CAR-NK-92MI monoclonal cell lines to CCRF-CEM cells. The cytotoxicity of 8 CD7-CAR-NK-92MI monoclonal cell lines to CCRF-CEM cells was detected under the condition of an effect-target ratio of 1:1 and co-culture for 24 hours. (C) The CAR positive rates of six bivalent dCD7-CAR-NK-92MI monoclonal cell lines were detected. (D) Cytotoxicity of six bivalent dCD7-CAR-NK-92MI monoclonal cell lines to CCRF-CEM cells. The cytotoxicity of six dCD7-CAR-NK-92MI monoclonal cell lines to CCRF-CEM cells was detected under the condition of an effect-target ratio of 1:1 and co-culture for 24 hours.

FIG. 5 shows a comparison of cytokine secretion of different monoclonal cells. (A) IFN-γ secretion of eight CD7-CAR-NK-92MI monoclonal cells after incubation with CCRF-CEM cells for 24 hours. (B) Granzyme B secretion of eight CD7-CAR-NK-92MI monoclonal cells after incubation with CCRF-CEM cells for 24 hours. (C) IFN-γ secretion of six dCD7-CAR-NK-92MI monoclonal cells after incubation with CCRF-CEM cells for 24 hours. (D) Granzyme B secretion of six dCD7-CAR-NK-92MI monoclonal cells after incubation with CCRF-CEM cells for 24 hours.

FIG. 6 shows that mdCD7-CAR-NK-92MI cells show effective anti-tumor activity in PDX model. (A) Schematic diagram of primary T-ALL xenotransplantation model. Mice were divided into two groups with 15 mice in each group. Mice in one group were injected with 2.0×10⁶ T-ALL cells (T-ALL dose 1), and mice in the other group were injected with 1.0×10⁷ T-ALL cells (T-ALL dose 2). After 3 days, the drugs were administered once every 2-4 days for a total of 5 injections. (B) Mean body weight of mice in low tumor burden group (T-ALL dose 1). (C) Survival curve of mice in low tumor burden group (T-ALL dose 1). n=5.** P<0.01. (D) Mean body weight of mice in high tumor burden group (T-ALL dose 2). (E) Survival curve of mice in high tumor burden group (T-ALL dose 2). n=5.** P<0.01. (F) Flow cytometry results of tumor burden in peripheral blood of mice treated with mdCD7-CAR-NK-92MI cells for 17 days. (G) Statistical analysis of tumor burden in peripheral blood after treatment with mdCD7-CAR-NK-92MI cells for 17 days. ** P<0.01, n=3.

FIG. 7 shows the cytotoxicity of tested NK-92MI, CD7-CAR-NK-92MI and dCD7-CAR-NK-92MI cells to Raji cells.

FIG. 8 shows that the mdCD7-CAR-NK-92MI monoclonal cell lines specifically target and eliminate primary CD7+T-ALL tumor cells (sample 2). (A) Expression level of CD7 in primary T-ALL tumor cells. (B) Cytotoxicity of mdCD7-CAR-NK-92MI cells to primary T-ALL tumor cells (Sample 2) under different effect-target ratios. 7-AAD negative cell population represents the percentage of remaining tumor cells. (C) After incubating mdCD7-CAR-NK-92MI monoclonal cells with T-ALL primary tumor cells for 24 hours, the concentration of IFN-γ in supernatant was detected. (D) After incubating mdCD7-CAR-NK-92MI monoclonal cells with T-ALL primary tumor cells for 24 hours, the concentration of granzyme B in supernatant was detected.

FIG. 9 shows the expansion of primary T-ALL tumor cells in mice. (A) A schematic diagram of the expansion of primary T-ALL tumor cells in mice. B-NSG mice were intravenously injected with 1×10⁷ T-ALL tumor cells. After 35 days, mice were killed and spleens were collected. (B) Flow cytometry analysis of T-ALL tumor cells in mouse spleen.

FIG. 10 shows a flow cytometry analysis of NK-92MI cell content in peripheral blood of mice three days after the last administration.

### DETAILED DESCRIPTION

Through extensive and intensive studies, the inventors unexpectedly discovered for the first time that CD7-CAR-NK-92MI and dCD7-CAR-NK-92MI cells can effectively treat T-cell leukemia. Specifically, the present inventor constructed monovalent CD7-CAR-NK-92MI and bivalent dCD7-CAR-NK-92MI cells based on CD7 nanobodies. CD7-CAR-NK-92MI and dCD7-CAR-NK-92MI cells showed specific and effective antitumor activity against T-cell leukemia cell lines and primary tumor cells, and bivalent mdCD7-CAR-NK-92MI monoclonal cells showed significant cytotoxicity to primary T-ALL tumor cells. Compared with control NK-92MI cells, mdCD7-CAR-NK-92MI cells have a significant increase in the release of interferon gamma and granzyme B after incubation with CD7-positive primary T-ALL cells. In addition, it was also found that mdCD7-CAR-NK-92MI cells can significantly inhibit tumor progression in xenograft mouse model of T-ALL primary cells. Therefore, the CD7-CAR-NK-92MI cells of the present invention can be used as a novel method for the treatment of T-cell acute lymphoblastic leukemia. On this basis, the present invention has been completed.

### The Terms

Unless otherwise defined, all technical and scientific terms used herein have the same meaning as commonly understood by those ordinarily skilled in the art to which this invention belongs.

As used herein, when referring to specifically recited values, the term “about” means that the value can vary from the recited value by no more than 1%. For example, as used herein, the expression “about 100” includes all values between 99 and 101 (e.g., 99.1, 99.2, 99.3, 99.4, etc.).

As used herein, the terms “contain” or “include (comprise)” may be open, semi-enclosed, and enclosed. In other words, the term also includes “substantially composed of”, or “composed of”.

Chimeric Antigen Receptor

The present invention provides a chimeric antigen receptor (CAR) comprising an extracellular domain, a transmembrane domain, and an intracellular domain. Extracellular domain includes target-specific binding element (also known as antigen binding domain). Intracellular domain includes costimulatory signal transduction region and zeta chain. Costimulatory signal transduction region refers to a part of intracellular domain including costimulatory molecules. Costimulatory molecules are cell surface molecules required for effective response of lymphocytes to antigens, rather than antigen receptors or their ligands.

Linkers may be incorporated between the extracellular domain and the transmembrane domain of the CAR, or between the cytoplasmic domain and the transmembrane domain of the CAR. As used herein, the term “linker” generally refers to any oligopeptide or polypeptide that functions to link a transmembrane domain to an extracellular domain or cytoplasmic domain of a polypeptide chain. The linker may comprise from 0 to 300 amino acids, preferably from 2 to 100 amino acids and most preferably from 3 to 50 amino acids.

In a preferred embodiment of the present invention, the extracellular domain of the CAR provided by the present invention comprises an antigen binding domain targeting CD7. When the CAR of the present invention is expressed in NK cells, it can perform antigen recognition based on the antigen binding specificity. When it binds to its associated antigen, it affects tumor cells, resulting in tumor cells not growing, being promoted to die or being affected in other ways, and causing the patient's tumor burden to shrink or eliminate. The antigen binding domain is preferably fused with an intracellular domain derived from one or more of the costimulatory molecule and the zeta chain. Preferably, the antigen binding domain is fused with the intracellular domain combined with the CD28 and/or 4-1BB signaling domain and the CD3 zeta signaling domain.

In another preferred embodiment, the structure of the CAR is as shown in Formula I:

L-S-H-TM-C-CD3ζ (I)

wherein, each “-” is independently a linking peptide or peptide bond;

L is an optional signal peptide sequence;

S is an antigen binding domain;

H is an optional hinge region;

TM is a transmembrane domain;

C is a co-stimulatory signal molecule;

CD3ζ is a cytoplasmic signal transduction sequence derived from CD3ζ.

In another preferred embodiment, the structure of the CAR is L-VHH-H-TM-C-CD3ζ or L-VHH-La-VHH-H-TM-C-CD3ζ, wherein each element is defined as described above.

In another preferred embodiment, the CAR has an amino acid sequence as shown in SEQ ID NO.: 3 or 4.

CD7-CAR amino acid sequence (SEQ ID NO.: 3):
(SEQ ID NO.: 3)
MLLLVTSLLLCELPHPAFLLIPMDVQLQESGGGLVQAGGSLRLSCAVSGY
PYSSYCMGWFRQAPGKEREGVAAIDSDGRTRYADSVKGRFTISQDNAKNT
LYLQMNRMKPEDTAMYYCAARFGPMGCVDLSTLSFGHWGQGTQVTVSITE
FGSESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKS
RWQEGNVFSCSVMHEALHNHYTQKSLSLSLGKMFWVLVVVGGVLACYSLL
VTVAFIIFWVRSKRSRGGHSDYMNMTPRRPGPTRKHYQPYAPPRDFAAYR
SKRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQ
ALPPR
dCD7-CAR amino acid sequence (SEQ ID NO.: 4):
(SEQ ID NO.: 4)
MLLLVTSLLLCELPHPAFLLIPMDVQLQESGGGLVQAGGSLRLSCAVSGY
PYSSYCMGWFRQAPGKEREGVAAIDSDGRTRYADSVKGRFTISQDNAKNT
LYLQMNRMKPEDTAMYYCAARFGPMGCVDLSTLSFGHWGQGTQVTVSITG
GGGSGGGGSGGGGSGGGGSMDVQLQESGGGLVQAGGSLRLSCAVSGYPYS
SYCMGWFRQAPGKEREGVAAIDSDGRTRYADSVKGRFTISQDNAKNTLYL
QMNRMKPEDTAMYYCAARFGPMGCVDLSTLSFGHWGQGTQVTVSITEFGS
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGKMFWVLVVVGGVLACYSLLVTV
AFIIFWVRSKRSRGGHSDYMNMTPRRPGPTRKHYQPYAPPRDFAAYRSKR
GRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

Antigen Binding Domain

In one embodiment, the CAR of the present invention comprises a target-specific binding element referred to as an antigen binding domain. The antigen binding domain of the CAR of the invention is a specific binding element targeting CD7, and the antigen binding domain contains a VHH sequence of nanobody targeting CD7.

In another preferred embodiment, the antigen binding domain contains n VHH sequences of nanobody targeting CD7, where n is a positive integer of 1-5, preferably n is a positive integer of 1-3, and more preferably n is 1 or 2.

In another preferred embodiment, when n≥2, the antigen binding domain also contains a linking peptide La between each VHH sequences of nanobody targeting CD7.

In another preferred embodiment, the linking peptide La has a length of 5-25, preferably 10-20 amino acids.

In another preferred embodiment, the structure of the antigen binding domain is V_{HH} or V_{HH}-I-V_{HH}, wherein the V_{HH} is the VHH sequence of nanobody targeting CD7, and I is none or the linking peptide La.

In another preferred embodiment, the antigen binding domain is one or two VHH sequences of nanobody targeting CD7.

In another preferred embodiment, the VHH sequence of the nanobody targeting CD7 is shown in SEQ ID NO.: 1.

(SEQ ID NO.: 1)
MDVQLQESGGGLVQAGGSLRLSCAVSGYPYSSYCMGWFRQAPGKEREGVA
AIDSDGRTRYADSVKGRFTISQDNAKNTLYLQMNRMKPEDTAMYYCAARF
GPMGCVDLSTLSFGHWGQGTQVTVSIT

In another preferred embodiment, the sequence of V_{HH}-I-V_{HH} is shown in SEQ ID NO.: 2.

(SEQ ID NO.: 2)
MDVQLQESGGGLVQAGGSLRLSCAVSGYPYSSYCMGWFRQAPGKEREGVA
AIDSDGRTRYADSVKGRFTISQDNAKNTLYLQMNRMKPEDTAMYYCAARF
GPMGCVDLSTLSFGHWGQGTQVTVSITGGGGSGGGGSGGGGSGGGGSMDV
QLQESGGGLVQAGGSLRLSCAVSGYPYSSYCMGWFRQAPGKEREGVAAID
SDGRTRYADSVKGRFTISQDNAKNTLYLQMNRMKPEDTAMYYCAARFGPM
GCVDLSTLSFGHWGQGTQVTVSIT

In another preferred embodiment, the sequence of the linking peptide La is shown in SEQ ID NO.: 5.

(SEQ ID NO.: 5)
GGGGSGGGGSGGGGSGGGGS

In another preferred embodiment, the antigen binding domain comprises a sequence as shown in SEQ ID NO.: 1 or 2.

In another preferred embodiment, the sequence of the antigen binding domain is as shown in SEQ ID NO.: 1 or 2.

In another preferred embodiment, the sequence of the antigen binding domain is of at least 70%, preferably at least 75%, 80%, 85%, 90%, more preferably at least 95%, 96%, 97%, 98% or more than 99% sequence identity to the sequence as shown in SEQ ID NO.: 1 or 2.

In another preferred embodiment, the antigen binding domain targets or binds to human CD7.

CD7

CD7 molecules are highly expressed on T-cell acute lymphoblastic leukemia (T-ALL) and about 10% of T-lymphocyte myeloid leukemia cells. CD7 is usually expressed in T-ALL and normal T lymphocytes, but it is not expressed in a small group of normal T lymphocytes. In addition, CD7 does not seem to have a critical effect on the development and function of T cells. Mouse T progenitor cells that have been destroyed the CD7 molecule will still produce normal T cell development and homeostasis. It only causes tiny T cell effector function. Therefore, CD7 may be a particularly suitable target for the treatment of T-ALL. However, the application of CD7-CAR-T still faces many challenges. Firstly, the expression of CD7 antigen by both T effector cells and T malignant tumors may lead to internecine killing of CD7-CAR-T cells. Secondly, collecting sufficient numbers of autologous T cells from relapsed and refractory patients without being contaminated by tumor cells is also technically challenging. In addition, although it has been recently reported that in mouse experiments, T-ALL can be eliminated by CD7-CAR-T allogeneic CD7-CAR-T cells that CD7 and T cell receptors are simultaneously knocked out, it is difficult to guarantee 100% knockout rate, and there may be the risk of graft-versus-host disease (GVHD) in clinical application.

The present invention constructs two CD7-CAR-NK-92MI cell lines (monovalent CD7-CAR-NK-92MI and bivalent dCD7-CAR-NK-92MI). The results show that these two kinds of CD7-CAR NK-92MI cells can specifically eliminate CD7-positive T-ALL cell lines and CD7-positive T-ALL primary tumor cells in vitro. The bivalent dCD7-CAR-NK-92MI monoclonal cell (mdCD7-CAR-NK-92MI cell) constructed by the present invention has effective anti-tumor effect in the mouse xenotransplantation model of T-ALL primary tumor cells, and significantly improves the overall survival rate of mice.

NK Cells

Natural killer (NK) cells are a class of major immune effector cells, which protect the body from virus infection and tumor cell invasion through non-antigen-specific pathways. In recent years, NK cells have shown great application prospects in adoptive cellular immunotherapy.

NK-92 cell is an interleukin-2 (IL-2)-dependent NK cell line derived from peripheral blood mononuclear cells of a 50-year-old white male patient with acute non-Hodgkin's lymphoma. NK-92 cells are currently the only NK cell line approved by the FDA for clinical trials. This cell line is highly cytotoxic, economical, off-the-shelf, easy to prepare on a large scale, and has a short survival time after killing tumor cells. It is easy to expand in vitro. The vast majority of treated patients do not reject NK-92 cells, have no risk of graft-versus-host reaction, do not express KIRs, and are in a constitutively activated state. It has shown good clinical safety so far.

NK92MI cell is an IL2 independent cell line derived from NK-92 cells obtained by transfection, which are cytotoxic to many malignant tumor cells and have a greater application prospect in clinical application. NK92MI cells are lymphocytes that can kill target cells without relying on antibody involvement, nor antigen stimulation and sensitization. It is cytotoxic to many malignant cells, and chromium release tests have shown that it can kill K562 and Daudi cells. NK92MI cells have been used in clinical research as an adoptive cellular immunotherapy cell, and have less side effects for patients with advanced cancer.

Natural killer (NK) cells play an important role in the innate immune defense against malignant cells, which makes them ideal effector cells for adoptive immunotherapy. The NK-92 cell line is a mononuclear cell line derived from the peripheral blood of a patient with non-Hodgkin's lymphoma. It is the only NK cell line that has been validated in clinical trials, and its safety has been verified in clinical trials of cancer and melanoma. NK-92 cells lack almost all inhibitory killer cell immunoglobulin-like receptors (KIR), but except for KIR2DL4, which inhibits NK cell activation by binding to human leukocyte antigen molecules on target cells. NK-92MI cells are derived from the NK-92 cell line and stably transfected with interleukin-2 (IL-2) gene to make them independent of IL-2, giving them the same characteristics as the parental NK-92 cells. CAR-modified NK cells deplete shortly after lysis of tumor cells. This feature avoids the need for an inducible safety switch when used in vivo. In addition, NK cells have been clinically observed to mediate anti-tumor effects, and there is almost no risk of graft-versus-host disease, and it has been verified in CAR applications and has been effective in several clinical trials.

In the present invention, unless otherwise specified, “NK cells”, “NK cells of the present invention” or “engineered NK cells” all refer to the NK cells according to the first aspect of the present invention. The NK cells express chimeric antigen receptor CAR, and the antigen binding domain of the CAR contains a CD7-targeting nanobody VHH sequence.

Vector

The present invention also provides a DNA construct encoding the CAR sequence of the invention.

The nucleic acid sequence encoding the desired molecule can be obtained by recombinant methods known in the art, for example, by screening a library from a cell expressing the gene, by obtaining the gene from a vector known to include the gene, or by directly isolating from cells and tissues containing the gene using standard techniques. Alternatively, the gene of interest can be synthesized and produced.

The present invention also provides a vector in which the DNA construct of the invention is inserted. Vectors derived from retroviruses such as lentivirus are suitable tools for long-term gene transfer because they allow long-term stable integration of transgenes and their proliferation in daughter cells. Lentiviral vectors have advantages over vectors derived from oncogenic retroviruses such as murine leukemia virus because they can transduce non-proliferating cells, such as hepatocytes. They also have the advantage of low immunogenicity.

In a simple summary, the expression of the natural or synthetic nucleic acid encoding the CAR is usually achieved by operably linking the nucleic acid encoding the CAR polypeptide or part thereof to the promoter, and incorporating the construct into an expression vector. The vector is suitable for replication and integration of eukaryotic cells. Typical cloning vectors contain transcriptional and translational terminators, initial sequences and promoters that can be used to regulate expression of desired nucleic acid sequences.

The expression constructs of the present invention can also utilize standard gene delivery schemes for nucleic acid immunization and gene therapy. Methods of gene delivery are known in the art. See, for example, U.S. Pat. Nos. 5,399,346, 5,580,859, 5,589,466, which are incorporated herein in its entirety by reference. In another embodiment, the present invention provides a gene therapy vector.

The nucleic acid can be cloned into many types of vectors. For example, the nucleic acid may be cloned into vectors including, but not limited to, plasmids, phage particles, phage derivatives, animal viruses, and clay particles. Specific vectors of interest include expression vectors, replication vectors, probe production vectors, and sequencing vectors.

Further, the expression vector can be provided to cells in the form of viral vectors. Viral vector techniques are well known in the art and are described, for example, in Sambrook et al. 2001, Molecular Cloning: A Laboratory Manual, Cold Spring Harbor Laboratory, New York and other manuals of virology and molecular biology. Viruses that can be used as vectors include, but are not limited to, retroviruses, adenoviruses, adeno-associated viruses, herpesviruses, and lentivirus. Typically, a suitable vector comprises a replication origin, a promoter sequence, a convenient restriction enzyme site, and one or more selectable markers that function in at least one organism (e.g., WO01/96584; WO01/29058; and U.S. Pat. No. 6,326,193).

Many virus-based systems have been developed for transferring genes into mammalian cells. For example, retroviruses provide a convenient platform for gene delivery systems. The selected gene can be inserted into a vector and packaged into retroviral particles using techniques known in the art. The recombinant virus can then be isolated and delivered to target cells in vivo or ex vivo. Many retroviral systems are known in the art. In some embodiments, adenoviral vectors are used. Many adenoviral vectors are known in the art. In one embodiment, lentiviral vectors are used.

Additional promoter elements, such as enhancers, can adjust the frequency of transcription initiation. Generally, these are located in the 30-110 bp region upstream of the start site, although it has recently been shown that many promoters also contain functional elements downstream of the start site. The spacing between promoter elements is often flexible to maintain promoter function when the element is inverted or moved relative to the other. In the thymidine kinase (tk) promoter, the spacing between promoter elements can be increased by 50 bp before the activity begins to decline. Depending on the promoter, it is shown that individual elements can act cooperatively or independently to initiate transcription.

An example of a suitable promoter is the immediate early cytomegalovirus (CMV) promoter sequence. The promoter sequence is a strong constitutive promoter sequence capable of driving high-level expression of any polynucleotide sequence operably linked to it. Another example of a suitable promoter is elongated growth factor-1 alpha (EF-1 alpha). However, other constitutive promoter sequences can also be used, including but not limited to the simian virus 40 (SV40) early promoter, mouse breast cancer virus (MMTV), human immunodeficiency virus (HIV) long terminal repeat (LTR) promoter, MoMuLV promoter, avian leukemia virus promoters, Epstein-Barr virus immediate early promoters, Rus' sarcoma virus promoters, and human gene promoters, such as but not limited to actin promoters, myosin promoter, heme promoter and creatine kinase promoter. Further, the present invention should not be limited to the application of constitutive promoters. Inducible promoters are also considered as part of the present invention. The use of inducible promoters provides molecular switches that can turn on expression of polynucleotide sequences that operably link to inducible promoters when such expression is desired or turn off expression when expression is undesirable. Examples of inducible promoters include but are not limited to metallothionein promoters, glucocorticoid promoters, progesterone promoters, and tetracycline promoters.

In order to evaluate the expression of the CAR polypeptide or part thereof, the expression vector introduced into the cell may also contain either or both of a selectable marker gene or a reporter gene, so as to facilitate the search for the cell population to be transfected or infected by the viral vector to identify and select expressing cells. In other aspects, selectable markers may be carried on a single piece of DNA and used in a co-transfection procedure. Both the selectable marker and the reporter gene can be flanked by appropriate regulatory sequences so that they can be expressed in the host cell. Useful selectable markers include, for example, antibiotic resistance genes, such as neo and the like.

Reporter genes are used to identify potentially transfected cells and to evaluate the functionality of regulatory sequences. Generally, a reporter gene is a gene that does not exist in or is expressed by a recipient organism or tissue, and it encodes a polypeptide whose expression is clearly indicated by some easily detectable properties such as enzyme activity. After the DNA has been introduced into the recipient cell, the expression of the reporter gene is measured at an appropriate time. Suitable reporter genes may include genes encoding luciferase, beta-galactosidase, chloramphenicol acetyltransferase, secretory alkaline phosphatase, or green fluorescent protein genes (e.g. Ui-Tei et al., 2000 FEBS Letters 479: 79-82). Suitable expression systems are well known and can be prepared using known techniques or commercially available. Generally, a construct with a minimum of 5 flanking regions that shows the highest level of reporter gene expression is identified as a promoter. Such promoter regions can be linked to reporter genes and used to evaluate the ability of reagents to regulate promoter-driven transcription.

Methods of introducing genes into cells and expressing genes into cells are known in the art. In the content of the expression vector, the vector can be easily introduced into a host cell by any method in the art, for example, a mammalian, bacterial, yeast, or insect cell. For example, the expression vector can be transferred into the host cell by physical, chemical or biological means.

Physical methods for introducing polynucleotides into host cells include calcium phosphate precipitation, lipofection, particle bombardment, microinjection, electroporation, and so on. Methods of producing cells including vectors and/or exogenous nucleic acids are well known in the art. See, for example, Sambrook et al. 2001, Molecular Cloning: A Laboratory Manual, Cold Spring Harbor Laboratory, New York. The preferred method for introducing polynucleotides into host cells is calcium phosphate transfection.

Biological methods for introducing polynucleotides of interest into host cells include the use of DNA and RNA vectors. Viral vectors, especially retroviral vectors, have become the most widely used method of inserting genes into mammalian, such as human cells. Other viral vectors may be derived from lentivirus, poxvirus, herpes simplex virus I, adenovirus and adeno-associated virus, etc. See, for example, U.S. Pat. Nos. 5,350,674 and 5,585,362.

Chemical means of introducing polynucleotides into host cells include colloidal dispersion systems, such as macromolecular complexes, nanocapsules, microspheres, beads; and lipid-based systems, including oil-in-water emulsions, micelles, mixed micelles and liposomes. Exemplary colloidal systems used as delivery vehicles in vitro and in vivo are liposomes (e.g., artificial membrane vesicles).

In the case of using a non-viral delivery system, an exemplary delivery tool is a liposome. Consider using lipid preparations to introduce nucleic acids into host cells (in vitro, ex vivo or in vivo). In another aspect, the nucleic acid may be associated with lipids. Lipid-associated nucleic acids can be encapsulated in the aqueous interior of liposomes, dispersed in the lipid bilayer of liposomes, and attached via linking molecules associated with both liposomes and oligonucleotides to liposomes, trapped in liposomes, complexed with liposomes, dispersed in a solution containing lipids, mixed with lipids, combined with lipids, contained in lipids as a suspension, contained in micelles or complexed with micelles, or in other ways to associated with lipids. The lipid, lipid/DNA or lipid/expression vector associated with the composition is not limited to any specific structure in the solution. For example, they may exist in bilayer structures, as micelles or have a “collapsed” structure. They can also be simply dispersed in solution, possibly forming aggregates of uneven size or shape. Lipids are fatty substances, which can be naturally occurring or synthesized lipids. Lipids, for example, include small droplets of fat that occur naturally in the cytoplasm and in compounds that contain long-chain aliphatic hydrocarbons and their derivatives such as fatty acids, alcohols, amines, amino alcohols, and aldehydes.

Therapeutic Application

The present invention includes cells (e.g., NK cells) transduced with lentiviral vectors (LV) encoding the CAR of the present invention. Transduced NK cells can induce CAR-mediated NK-cell response.

Therefore, the present invention also provides a method for stimulating a T cell-mediated immune response to a target cell population or tissue of a mammal, which comprises the following steps: administering the NK cells expressing the CAR of the present invention to the mammal.

In one embodiment, the present invention includes a class of cell therapies in which NK cells are genetically modified to express the CAR of the present invention and CAR-NK cells are injected into a recipient in need thereof. The injected cells can kill the tumor cells of the recipient. Unlike antibody therapy, CAR-NK cells can replicate in vivo, producing long-term persistence that can lead to sustained tumor control.

In one embodiment, the CAR-NK cells of the present invention can undergo stable in vivo T cell expansion and can continue for an extended amount of time. In addition, the CAR-mediated immune response can be part of an adoptive immunotherapy step in which CAR-modified NK cells induce an immune response specific to the antigen-binding domain in the CAR. For example, anti-CD7 CAR-NK cells elicit a specific immune response against CD7-expressing cells.

Although the data disclosed herein specifically discloses a lentiviral vector including anti-CD7 nanobody, human Fc hinge region, CD28 transmembrane region and intracellular region, and 4-1BB and CD3ζ signaling domains, the present invention should be explained to include any number of changes to each of the components of the construct.

Treatable cancers include tumors that have not been vascularized or have not been substantially vascularized, as well as vascularized tumors. Cancers may include non-solid tumors (such as hematological tumors such as leukemia and lymphoma) or may include solid tumors. Types of cancer treated with the CAR of the present invention include, but are not limited to, carcinomas, blastocytomas and sarcomas, and certain leukemia or lymphoid malignancies, benign and malignant tumors, and malignant tumors, such as sarcomas, carcinomas and melanomas. It also includes adult tumors/cancers and childhood tumors/cancers.

Hematologic cancer is a cancer of the blood or bone marrow. Examples of hematological (or hematogenic) cancers include leukemias, including acute leukemias (such as acute lymphoblastic leukemia, acute myeloid leukemia, acute myeloid leukemia and myeloblastic, promyelocytic, myelomonocytic type, monocytic and erythroleukemia), chronic leukemia (such as chronic myeloid (granulocyte) leukemia, chronic myelogenous leukemia, and chronic lymphocytic leukemia), polycythemia vera, lymphoma, Hodgkin's disease, non-Hodgkin's lymphoma (painless and high-grade form), multiple myeloma, Waldenstrom's macroglobulinemia, heavy chain disease, myelodysplastic syndrome, hairy cell leukemia, and myelodysplasia.

Solid tumors are abnormal masses of tissue that usually do not contain cysts or fluid areas. Solid tumors can be benign or malignant. Different types of solid tumors are named after the cell types that form them (such as sarcoma, cancer and lymphoma). Examples of solid tumors such as sarcoma and cancer include fibrosarcoma, myxosarcoma, liposarcoma, mesothelioma, lymphoid malignancies, pancreatic cancer, and ovarian cancer.

The CAR-modified NK cells of the present invention can also be used as a type of vaccine for ex vivo immunity and/or in vivo therapy of mammals. Preferably, the mammal is a human.

For ex vivo immunization, at least one of the following occurs in vitro before administering the cells into the mammal: i) expanding the cells, ii) introducing the CAR-encoding nucleic acid into the cells, and/or iii) cryopreserving the cells.

Ex vivo procedures are well known in the art and are discussed more fully below. Briefly, cells are isolated from mammals (preferably humans) and genetically modified (i.e., transduced or transfected in vitro) with a vector expressing the CAR disclosed herein. CAR-modified cells can be administered to mammalian recipients to provide therapeutic benefits. The mammalian recipient may be a human, and the CAR-modified cells may be autologous relative to the recipient. Alternatively, the cells may be allogeneic, syngeneic, or xenogeneic relative to the recipient.

In addition to using cell-based vaccines for ex vivo immunization, the present invention also provides compositions and methods for in vivo immunization to elicit an immune response against an antigen in a patient.

Generally, cells activated and expanded as described herein can be used to treat and prevent diseases that develop in individuals without an immune response. In particular, the CAR-modified NK cells of the present invention are used for the treatment of T-ALL. In certain embodiments, the cells of the present invention are used to treat patients at risk of developing T-ALL. Thus, the present invention provides a method of treating or preventing T-ALL, which comprises administering a therapeutically effective amount of CAR-modified NK cells of the present invention to a subject in need thereof.

The CAR-modified NK cells of the present invention can be administered alone or as a pharmaceutical composition in combination with a diluent and/or other components such as IL-2, IL-17 or other cytokines or cell populations. Briefly, the pharmaceutical composition of the present invention may include the target cell population as described herein, in combination with one or more pharmaceutically or physiologically acceptable carriers, diluents or excipients. Such compositions may include buffers such as neutral buffered saline, sulfate buffered saline, etc.; carbohydrates such as glucose, mannose, sucrose or dextran, mannitol; proteins; polypeptides or amino acids such as glycine; antioxidants; chelate agents such as EDTA or glutathione; adjuvants (for example, aluminum hydroxide); and preservatives. The compositions of the present invention are preferably formulated for intravenous administration.

The pharmaceutical composition of the present invention can be administered in a manner suitable for the disease to be treated (or prevented). The number and frequency of administration will be determined by factors such as the patient's condition, and the type and severity of the patient's disease—although the appropriate dosage can be determined by clinical trials.

When “immunologically effective amount”, “anti-tumor effective amount”, “tumor-inhibitory effective amount” or “therapeutic amount” is indicated, the precise amount of the composition of the present invention to be administered can be determined by the physician, who considers the age, weight, tumor size, degree of infection or metastasis and individual differences in the condition of the patient (subject). It may generally be noted that a pharmaceutical composition comprising the T cells described herein can be administered at a dose of 10⁴ to 10 cells/kg body weight, preferredly at a dose of 10⁵ to 10⁶ cells/kg body weight (including all integer values within those ranges). The T cell composition can also be administered multiple times at these doses. The cells can be administered by using injection techniques known in immunotherapy (see, for example, Rosenberg et al., New Eng. J. of Med. 319:1676, 1988). The optimal dosage and treatment regimen for a specific patient can be easily determined by those skilled in the medical field by monitoring the patient's signs of disease and adjusted accordingly.

The administration of the subject composition can be carried out in any convenient manner, including by spraying, injection, swallowing, infusion, implantation, or transplantation. The compositions described herein can be administered to patients subcutaneously, intracutaneously, intratumorally, intranodal, intraspinal, intramuscular, by intravenous (i.v.) injection, or intraperitoneally. In one embodiment, the T cell composition of the present invention is administered to a patient by intradermal or subcutaneous injection. In another embodiment, the T cell composition of the present invention is preferably administered by i.v. injection. The composition of T cells can be directly injected into tumors, lymph nodes or infected sites.

In some embodiments of the present invention, the cells activated and expanded using the methods described herein or other methods known in the art to expand T cells to therapeutic levels are administered to the patient in combination with any number of relevant forms of treatment (e.g., before, simultaneously, or after). The forms of treatment include, but are not limited to, treatment with the following agents: the agents such as antiviral therapy, cidofovir and interleukin-2, cytarabine (also known as ARA-C) or Natalizumab treatment for MS patients or irfaizumab treatment for psoriasis patients or other treatments for PML patients. In a further embodiment, the T cells of the present invention can be used in combination with chemotherapy, radiation, immunosuppressive agents, such as cyclosporine, azathioprine, methotrexate, mycophenolate mofetil, and FK506, antibodies or other immunotherapeutics. In a further embodiment, the cell composition of the present invention is administered to a patient in combination with a bone marrow transplantation using chemotherapeutic agents such as fludarabine, external beam radiotherapy (XRT), cyclophosphamide (for example, before, simultaneously, or after). For example, in one embodiment, the subject may undergo the standard treatment of high-dose chemotherapy followed by peripheral blood stem cell transplantation. In some embodiments, after transplantation, the subject receives an infusion of the expanded immune cells of the present invention. In an additional embodiment, the expanded cells are administered before or after surgery.

The dose of the above treatment administered to the patient will vary depending on the precise nature of the treatment condition and the recipient of the treatment. The proportion of doses administered to humans can be implemented according to accepted practices in the art. Usually, for each treatment or each course of treatment, 1×10⁶ to 1×10¹⁰ modified CAR-NK cells of the present invention (e.g. CD7-CAR-NK cells) are administered to a patient, e.g., by intravenous reinfusion.

### The Technical Scheme of the Present Invention has the Following Beneficial Effects

1. The engineered NK cell specifically targets CD7 and can effectively kill tumor cells, especially has significant cytotoxicity to T cell tumors (such as primary T-ALL tumor cells), and has very effective therapeutic effect on T cell leukemia (such as T-ALL).

2. The present invention selects the nanobody as the antigen binding domain of the CAR. Compared with the traditional CAR containing scFv, the CAR of the present invention is easier to transfect T cells and has lower immunogenicity.

The CAR prepared based on a specific anti-CD7 nanotibody (SEQ ID NO.: 1) can kill tumor cells more specifically and effectively, especially T tumor cells.

4. Compared with unmodified NK cells, the secretion of interferon gamma and granzyme B of the engineered NK cells of the present invention is significantly improved. It has synergistic effect with CAR targeting CD7 on NK cells, thus killing tumor cells more effectively.

5. Compared with CAR-T cells, the engineered NK cells of the present invention can directly kill tumor cells by releasing granzyme, and since it lasts for a shorter time in the body, fewer cytokines are released, which reducing the risk of cytokine storms and making it safer.

6. The NK cells of the present invention are general-purpose cells, which can be developed into “off-the-shelf” products and can be prepared on a large scale, with uniform and stable quality, and can be used at any time for any patient. Moreover, GVHD and HVG can be avoided, treatment costs can be reduced, and immunotherapy side effects can be reduced. And NK cells do not come from patients, so there is no potential pollution risk.

7. Bivalent DCD7-CAR NK-92MI cells are significantly cytotoxic to primary T-ALL tumor cells and can significantly inhibit tumor progression in a xenograft mouse model of primary T-ALL cells, producing more cytokines after incubation with tumor cells. Compared with monovalent CD7-CAR NK-92MI cells, it has stronger killing activity to tumor cells. Compared with CAR T cells, it has a better safety profile due to its short survival time in vivo.

The invention is further illustrated below in conjunction with specific embodiments. It should be understood that the examples are not intended to limit the scope of the invention. The experimental methods in the following examples which do not specify the specific conditions are usually in accordance with conventional conditions, such as conditions described in Sambrook et al., Molecular Cloning: Laboratory Manual (New York: Cold Spring Harbor Laboratory Press, 1989), or in accordance with the conditions recommended by the manufacturer. Unless otherwise stated, percentages and parts are calculated by weight.

Materials and Methods

1 Cell Lines and Primary Tumor Cells

CD7-positive Jurkat and CCRF-CEM leukemia cell lines and CD7-negative Raji lymphoblastic cell lines were all purchased from the American Type Culture Collection (ATCC; Manassas, Va., USA). NK-92MI cell line expressing human IL-2 was also purchased from ATCC. T-ALL primary tumor cells were provided by the Department of Hematology, Jiangsu Provincial Hospital of Traditional Chinese Medicine. The Jurkat, CCRF-CEM, Raji and T-ALL primary cells were all cultured in RPMI-1640 medium (Hyclone) containing 10% fetal bovine serum (Gibco). The culture medium of all cells used in this study contained 0.1 mg/mL streptomycin (Gibco) and 100 U/mL penicillin (Gibco). The cells were cultured in a carbon dioxide incubator containing 5% carbon dioxide at 37° C.

2 Construction of CD7-Specific CAR Vector

The present invention constructs two CD7-CAR vectors using CD7 nanobody sequence monovalent VHH6 and bivalent VHH6-VHH6. The sequence of the CD7 nanobody is as shown in SEQ ID NO.: 1. The structure of monovalent CD7-CAR or divalent dCD7-CAR consists of signal peptide, monovalent CD7 nanobody sequence VHH6 or divalent CD7 nanobody sequence VHH6-VHH6, Fc hinge, CD28 transmembrane and intracellular domain, 4-1BB and CD3ζ intracellular domain, respectively. The sequence of the monovalent CD7-CAR is shown as SEQ ID NO.: 3, and the sequence of the bivalent dCD7-CAR is shown as SEQ ID NO.: 4. Then the coding sequences of CD7-CAR and dCD7-CAR were subcloned into the pHULK PiggyBac electrotransformation expression vector, the cloning sites were XbaI and EcoRI sites, and the constructed plasmids were named CD7-CAR plasmid and dCD7-CAR plasmid, respectively.

3 Construction of CD7-Specific CAR Modified NK-92MI Cells (CD7-CAR-NK-92MI)

In order to construct CD7-CAR-NK-92MI and dCD7-CAR-NK-92MI stable cell lines, the number of NK-92MI (ATCC, USA) cells was counted, and 1*10⁶ of them were electroporated. 5 ug of the VHH6-CAR and dVHH6-CAR electrotransformation plasmids were added to the electroporation cup (Catalog #: VCA-1001, Lonza, Germany). The electroporator Lonza 2b (Lonza, Germany) and the electroporation solution (Catalog #: VCA-1001, Lonza, Germany) with a volume of 100 ul for electroporation were selected with electroporation program U14 for electroporation; after electroporation, cells were placed in a 6-well plate (Labserv, Fisher Scientific, USA) with MEM-α (gibco, California) for recovery culture; when the cell state was restored, flow cytometry was performed, and 1 ug/ml puromycin (Acros, Belgium) was added for selection and culture to ensure the stable expression of CD7-CAR.

4 Flow Cytometry Analysis

In order to detect the expression of CD7-CAR or dCD7-CAR on the surface of NK-92MI cells, APC-conjugated anti-human IgG Fc antibody was incubated with NK-92MI, CD7-CAR-NK-92MI and dCD7-CAR-NK-92MI cells, respectively. In order to detect the expression of CD7 antigen on the cell surface, APC-conjugated anti-CD7 antibody (Becton Dickinson) was used to be incubated together with Jurkat, CCRF-CEM, Raji, T-ALL primary tumor cells, NK-92MI, CD7-CAR-NK-92MI and dCD7-CAR-NK-92MI cells, respectively. The cells and the antibody were incubated at 37° C. for 15 minutes, washed with phosphate buffered saline (PBS) three times, and then detected and analyzed using a flow cytometer (BD Biosciences).

5 Western Blot

1) VHH6-NK-92MI, dVHH6-NK-92MI and NK-92MI cells were collected as negative controls, respectively.

2) The collected cells were centrifuged, and then washed with PBS for 3 times. 50 μL of protein lysate was added to each tube, then 10 μL of 5× protein loading solution was added and protein denaturation was performed at 100° C. for 10 min.

3) Electrophoresis. 5 μL protein marker was added, and 10 μL sample was added to each well. Upper gel electrophoresis was at 80V for 30 min; the lower gel electrophoresis was at 120V for about 120 min.

4) Transmembrance. After electrophoresis, PVDF membrane was soaked in anhydrous methanol for 5 min. The transmembrance was started at 400 mA for 90 min. Electrode plates placement order: white splint (positive)-sponge-filter paper-PVDF membrane-protein gel-filter paper-sponge-black splint (negative).

5) Blocking. After transmembrance, the PVDF membrane was fixed with methanol for 3 min. Then it was sealed with 5% skimmed milk prepared in advance, and sealed on a shaker for 1 hour.

6) Primary antibody incubation. After blocking, the primary antibody was incubated at 4° C. overnight. The primary antibody was mouse anti-human CD3ζ, which was diluted with 5% skim milk at a concentration of 1:1000.

7) The overnight PVDF membrane was washed with 1×TBST buffer for 3 times, each time for 10 min.

8) The secondary antibody was incubated for 2 h at room temperature. The secondary antibody was HRP-goat anti-mouse IgG (H+L), which was diluted with 5% skimmed milk at a concentration of 1:10000.

9) PVDF membrane was rinsed with 1×TBST buffer three times for 10 min each time. The developing solution was added to the membrance, and the membrance was developed in the darkroom and the exposure time was 3 min.

6 Cytotoxicity Assay

The CFSE/7-AAD flow cytometry assay was used to test the cytotoxicity of CD7-CAR-NK-92MI or dCD7-CAR-NK-92MI cells to T-ALL tumor cell lines or primary tumor cells. Target cells (Jurkat, CCRF-CEM, Raji, T-ALL primary tumor cells) were re-suspended in PBS and treated with 1 μL carboxyfluorescein succinimide ester (CFSE) at 37° C. for 30 minutes. Raji cells were used as negative target cells in control group. The effector cells and target cells were added to the 24-well plate according to different ratio of effector to target and incubated for 4 or 24 hours. Cells were then collected and suspended in equal volume of PBS and 7-AAD (7-aminoactinomycin D; BD) was added. The percentage of specific lysis target cells (CFSE positive and 7-AAD positive) was detected by flow cytometry. CFSE positive cells are target cells, the percentage of 7-AAD positive cells reflects the mortality of target cells, and the percentage of 7-AAD negative cells reflects the percentage of remaining target cells.

7 CD7-CAR-NK-92MI and dCD7-CAR-NK-92MI Monoclonal Screening

Mouse trophoblast cells (5×10³) were added to each well of a 96-well plate, and each well contained 200 μl MEM-α medium. CD7-CAR-NK-92MI or dCD7-CAR-NK-92MI cells were screened by FACSAria TM III cell sorter (BD Biosciences, NJ) and APC-conjugated anti-human IgG Fc antibody. Then, the monoclonal cells were co-cultured in a 96-well plate containing mouse trophoblast cells. After about 28 weeks, the expansion of monoclonal cells in the 48-well plate was observed under microscope. Finally, the CAR positive rate of monoclonal cells was detected by flow cytometry (BD Biosciences).

8 Cytotoxicity of Different Monoclonal Cells

CCRF-CEM cells (CD7 positive cells) were selected as the target cells for killing experiment, and different monovalent or bivalent CD7-CAR-NK-92MI monoclonal cells were used as effector cells. The experimental method is the same as the above (2.6 cytotoxicity assay). After 24 hours of co-cultivation with an effector target ratio of 1:1, flow cytometry analysis was performed to screen the most active monoclonal cell line.

9 Cytokine Secretion of Different Monoclonal Cells

CD7-NK-92MI and dCD7-NK-92MI cells were incubated with CCRF-CEM cells (CD7 positive tumor cells) to produce interferon γ (IFN-γ) and granzyme B secretion, which was detected by CBA (cytometric bead array) detection kit. Effector cells were co-cultured with a constant number of target cells (2×10⁵) in a 24-well microculture plate at an effector-target ratio of 1:1, and the final volume was 1 ml of RPMI 1640 complete medium. After incubation for 24 hours, the supernatant was collected and determined by CBA kit. The Human Granzyme B CBA Flex Set D7 Kit (catalog No. 560304) and the Human IFN-γ CBAFlex Set E7 Kit (catalog No. 558269) were purchased from BD Corporation.

10 Cytotoxicity of Bivalent mdCD7-CAR-NK-92MI Monoclonal Cells to T-ALL Primary Tumor Cells

The cytotoxic activity of bivalent CD7-CAR NK-92MI monoclonal cells (mdCD7-CAR NK-92MI) against T-ALL primary tumor cells was detected in vitro based on cytokine secretion. T-ALL primary tumor cells were provided by the Department of Hematology, Jiangsu Provincial Hospital of Traditional Chinese Medicine. T-ALL primary tumor cells were labeled with CFSE at 37° C. for 30 minutes, seeded in a 24-well plate, and then mdCD7-CAR-NK92MI cells were added at different E:T ratios to co-culture with target cells. After 24 hours of incubation, the supernatant was collected, and the secretion of IFN-γ and granzyme B was analyzed with a CBA kit. The target cells were collected and resuspended in PBS containing 1μ of 7-aminoactinomycin D (7-AAD; BD). Then FACSCalibur flow cytometer (BD) was used for detection.

11 Animal Experiments

Female NOD-PrkdcscidIl2rgtm1/Bcgen (B-NSG) mice (Biocytogen) aged 6 to 7 weeks were injected with T-ALL primary tumor cells through tail vein. Three B-NSG mice were injected with 1×10⁷ cells each mouse, and they were euthanized when they were dying. The spleen cells of mice were collected and treated with erythrocyte lysate, and the proportion of T-ALL cells was detected by cell flow cytometer. The obtained T-ALL cells were transferred to 30 mice which were divided into two groups (low tumor burden group and high tumor burden group), with 15 mice in each group. Each mouse in the low tumor burden group was injected with 2×10⁶ T-ALL cells, and each mouse in the high tumor burden group were injected with 1×10⁷ T-ALL cells. Mice in low or high tumor burden groups were divided into three groups: (i) intravenous injection of PBS (n=5), (ii) intravenous injection of 1×10⁷ 60Co-irradiated (10Gy) NK-92MI cells (n=5), and (iii) intravenous injection of 1×10⁷ 60Co-irradiated (10Gy) mdCD7-NK-92MI cells (n=5). The drug was administered three days after the tumor cells were injected, and every two to four days, for a total of five doses. Three days after the last administration, tumor burden and the residual status of nK-92MI cells in peripheral blood were detected after orbital blood sampling.

### Example 1 Construction of CD7-CAR/dCD7-CAR Vectors and CAR-NK-92MI Cells

Two CD7-CARs were constructed using VHH6 monovalent (SEQ ID NO.: 1) and bivalent (SEQ ID NO.: 2) sequences of CD7 nanobody. Monovalent CD7-CAR consists of a signal peptide, an anti-CD7 nanobody sequence (VHH6), a human Fc hinge region, a CD28 transmembrane domain and a CD28 and 4-1BB intracellular signaling domain in tandem with the CD3ζ signaling domain. The bivalent dCD7-CAR contains a signal peptide, an anti-CD7 nanobody repeat sequence (VHH6-VHH6), a human Fc hinge region, a CD28 transmembrane domain, and a CD28 and 4-1BB intracellular signaling in tandem with CD3ζ signaling domain.

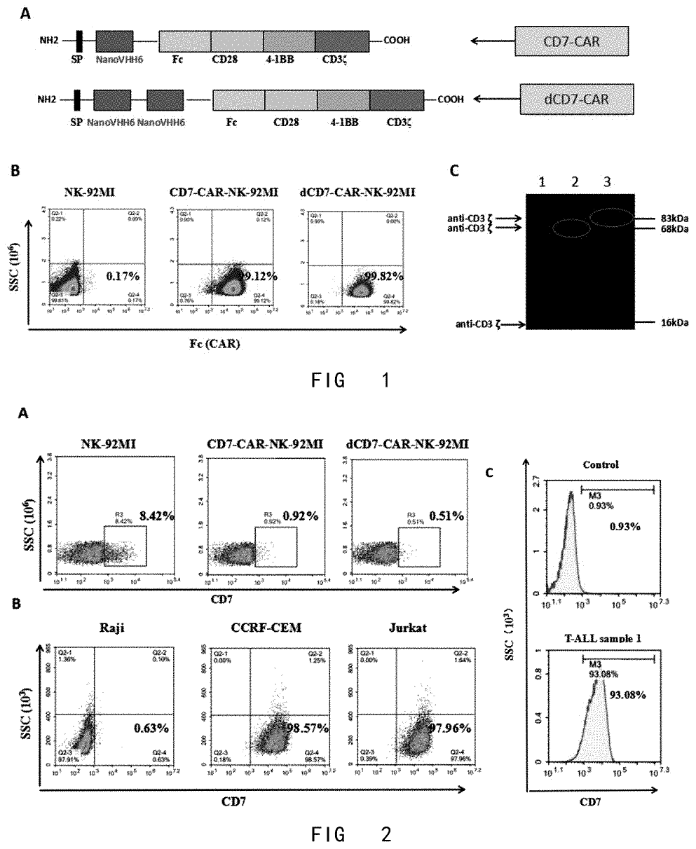

The structural diagram of CAR is shown in FIG. 1A. CD7-CAR and dCD7-CAR were cloned into pHULK PiggyBac electransformation expression vectors and named as CD7-CAR plasmids and dCD7-CAR plasmids respectively.

After electrotransduction, CD7-CAR-NK-92MI and dCD7-CAR-NK-92MI cells were sorted by flow cytometry with anti-Fc antibody. After sorting, the cells were cultured in a medium containing puromycin (1 μg/ml) for 3-4 months to obtain stable cell lines. The CAR protein expression on the cell surface of CD7-CAR-NK-92MI and dCD7-CAR-NK-92MI cells was detected by flow cytometry. The result shows that the positive rate is about 99% (FIG. 1B).

The expression of CD7-CAR and dCD7-CAR fusion proteins was also detected by Western blot in this example (FIG. 1C). Under deformation conditions, endogenous CD3ζ was expressed in the lysates of NK-92MI (lane 1), CD7-CAR-NK-92MI (lane 2) and dCD7-CAR-NK-92MI (lane 3), with a 16-kDa band (the size of CD3ζ endogenous protein). However, only two other larger bands were observed in CD7-CAR-NK-92MI and dCD7-CAR-NK-92MI cells, and were consistent with the theoretical size of monovalent CD7-CAR fusion protein or bivalent dCD7 (68-kDa or 83-kDa) (FIG. 1C).

### Example 2 Expression of CD7 in NK-92MI and T-ALL Cells

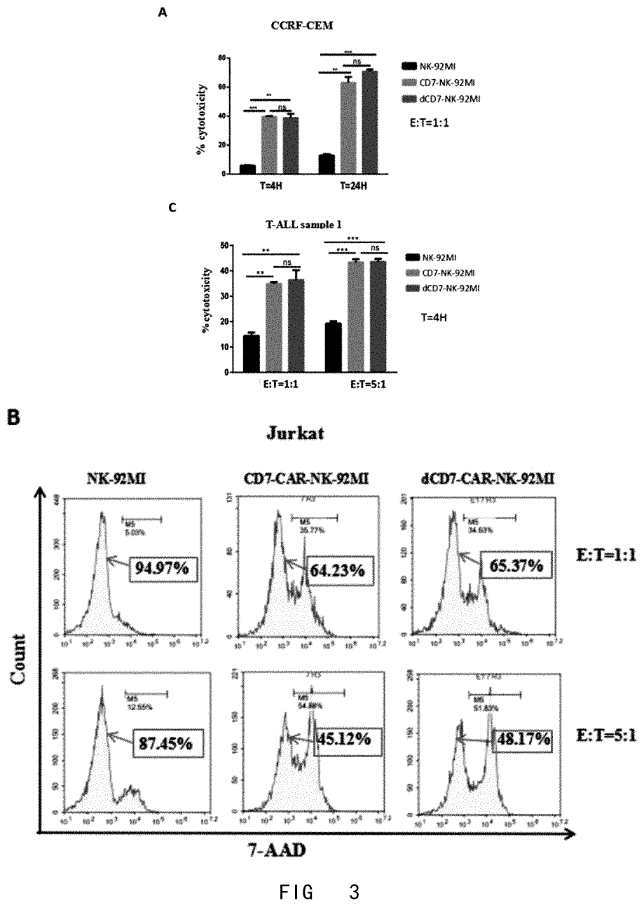

The expression of CD7 in NK-92MI, CD7-CAR-NK-92MI and dCD7-CAR-NK-92MI cells was detected by flow cytometry. The results shows that the positive rate of CD7 in NK-92MI cells is 8.42%, while the positive rate of CD7 in NK-92MI cells transfected with CD7-CAR (dCD7-CAR) is less than 1% (FIG. 2A). These results indicate that CD7-CAR-NK-92MI or dCD7-CAR-NK-92MI cells can kill CD7 positive NK-92MI cells specifically. The expression of CD7 on the surface of leukemia cell lines (Jurkat and CCRF-CEM), lymphoblastic cell lines (Raji) and primary tumor cells from T-ALL was also detected in this example. The results show that the CD7 positive rate in CCRF-CEM and Jurkat cells is almost 100% (FIG. 2B), the CD7 positive rate in T-ALL primary tumor cells is 93% (FIG. 2C, the control group is T-ALL cells that have not been incubated with CD7 antibody), and Raji cells are CD7 negative cells (FIG. 2B).

### Example 3 Killing Activity of CD7-CAR-NK-92MI and dCD7-CAR-NK-92MI Cells Against T-ALL Cell Line In Vitro

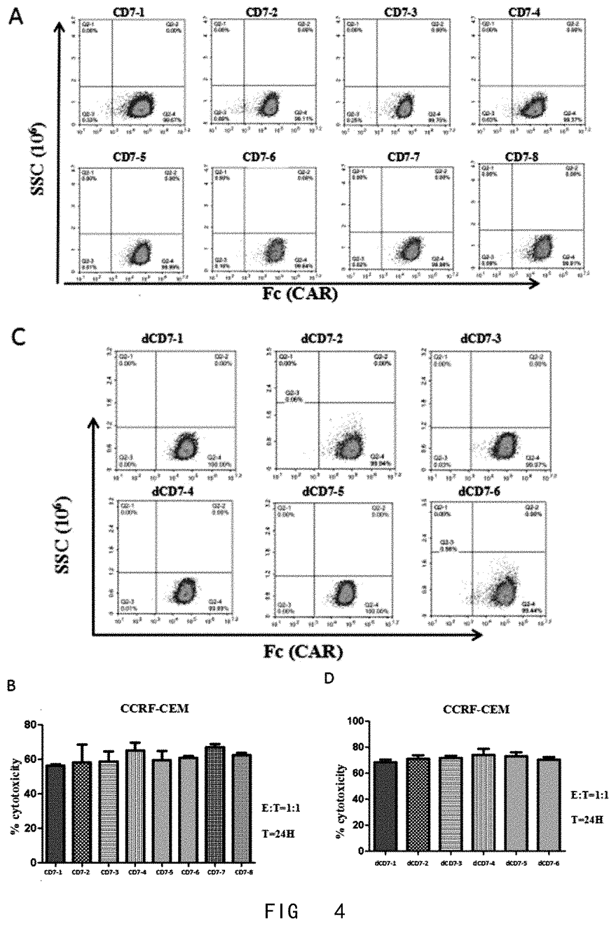

CD7 positive T-ALL cell lines (CCRF-CEM and Jurkat cells) were used to evaluate the antitumor activity of CD7-CAR-NK92-MI and dCD7-CAR-NK92-MI cells in vitro. Raji cells were used as negative cells lines. The cytotoxicity of CD7-CAR-NK92-MI and dCD7-CAR-NK92-MI cells to CCRF-CEM cells was detected by flow cytometry after co-culture with CCRF-CEM cells for 4 hours or 24 hours (the ratio of effector to target was 1:1). The results show that CD7-CAR-NK-92MI and dCD7-CAR-NK-92MI cells show significant specific cytotoxicity to CCRF-CEM cells under different effector-target ratios compared with control NK92-MI cells (FIG. 3A). This example also evaluated the cytotoxicity of CD7-CAR-NK-92MI and dCD7-CAR-NK-92MI cells to Jurkat cells at effector-target ratios of 1:1 and 5:1. The results show that after 24 hours of incubation with Jurkat cells, the percentage of remaining tumor cells in the CD7-CAR-NK-92MI group and dCD7-CAR-NK-92MI group is significantly lower than that in the control NK-92MI group (FIG. 3B). Compared with control NK-92MI cells, CD7-CAR-NK-92MI and dCD7-CAR-NK-92MI have stronger cytotoxicity to Jurkat cells. However, CD7-CAR-NK-92MI or dCD7-CAR-NK92-MI do not show specific cytotoxicity to Raji cells at an E-T cell ratio of 5:1 compared to control NK-92MI cells (FIG. 7). These results indicate that CD7-CAR NK-92MI and DCD7-CAR NK-92MI only have specific cytotoxicity to CD7-positive tumor cells.

### Example 4 Anti-Tumor Effects of CD7-CAR-NK-92MI and dCD7-CAR-NK-92MI on T-ALL Primary Tumor Cells

In order to further evaluate the anti-tumor effects of CD7-CAR-NK-92MI and dCD7-CAR-NK-92MI cells, their cytotoxicity to primary T-ALL tumor cells was tested. The CD7 positive rate of T-ALL primary tumor cells was 93% (FIG. 2C). The results show that CD7-CAR-NK-92MI and dCD7-CAR-NK-92MI cells have significant specific cytotoxicity to primary T-ALL tumor cells under the conditions of effector-target ratio of 1:1 and 5:1, compared with control NK-92MI cells (FIG. 3C). The results show that CD7-CAR-NK-92MI and dCD7-CAR-NK-92MI have specific cytotoxicity to T-ALL cell lines and primary T-ALL tumor cells in vitro.

### Example 5 Comparison of Activity Between Different Monovalent CD7-CAR-NK-92MI and Divalent dCD7-CAR-NK-92MI Monoclonal Cells

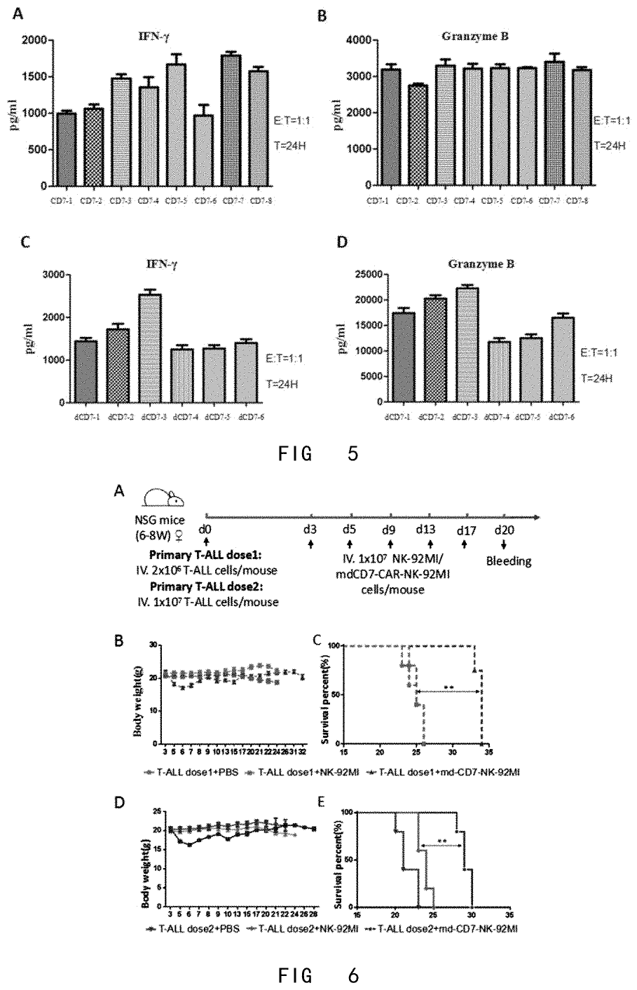

CD7-CAR-NK-92MI and dCD7-CAR-NK-92MI cells were further screened in order to maintain the stable expression of CAR in long-term culture. In this example, 8 monovalent CD7-CAR-NK-92MI monoclonal cell lines were finally screened, and the CD7-CAR positive rate of 8 monoclonal cell lines was close to 100% (FIG. 4A). CCRF-CEM cells were used as target cells, and the killing activities of eight monoclonal cells against CCRF-CEM cells were compared in vitro (FIG. 4B). The results show that eight monovalent CD7-CAR-NK-92MI monoclonal cell lines show significant cytotoxicity to CCRF-CEM cells at the ratio of effector to target of 1:1, but there is no significant difference in cytotoxicity among the 8 monoclonal cells. Then, the secretion of IFN-γ and granzyme B of 8 monovalent CD7-CAR-NK-92MI monoclonal cells incubated with CCRF-CEM was analyzed (FIGS. 5A and 5B). The results show that there are significant differences in the release of IFN-γ in different monoclonal cell lines, but there is no significant difference in the release of granzyme B. In addition, six bivalent dCD7-CAR-NK-92MI monoclonal cell lines were screened, and the CD7-CAR positive rate of the six monoclonal cell lines was close to 100% (FIG. 4C). Six bivalent dCD7-CAR-NK-92MI monoclonal cell lines also showed significant cytotoxicity to CCRF-CEM cells at an effector-target ratio of 1:1 (FIG. 4D). Similarly, the secretion of IFN-γ and granzyme B of six bivalent dCD7-CAR-NK-92MI monoclonal cells incubated with CCRF-CEM cells was also analyzed (FIGS. 5C and 5D). The results show that the release levels of IFN-γ and granzyme B in different bivalent monoclonal cells are significantly different.

These results indicate that although the cytotoxicity of monovalent CD7-CAR-NK-92MI and bivalent dCD7-CAR-NK-92MI cells to CCRF-CEM cells is very similar, the cytotoxicity of dCD7-CAR-NK-92MI cells is slightly stronger (FIGS. 4B and 4D). The release of IFN-γ and granzyme B is different in different monoclonal cell lines (FIG. 5). The ability of bivalent dCD7-CAR-NK-92MI monoclonal cells to secrete granzyme B is significantly higher than that of monovalent CD7-CAR-NK-92MI monoclonal cells (FIG. 5B, 5D). The dCD7-3 monoclonal cells in the bivalent DCD7-CAR-NK-92MI monoclonal cells have the strongest cytokine secretion capacity among all monoclonal cells. The bivalent dCD7-3 monoclonal cell line is named as mdCD7-CAR-NK-92MI cell.

### Example 6 Cytotoxicity of mdCD7-CAR-NK-92MI Cells to Primary T-ALL Tumor Cells In Vitro

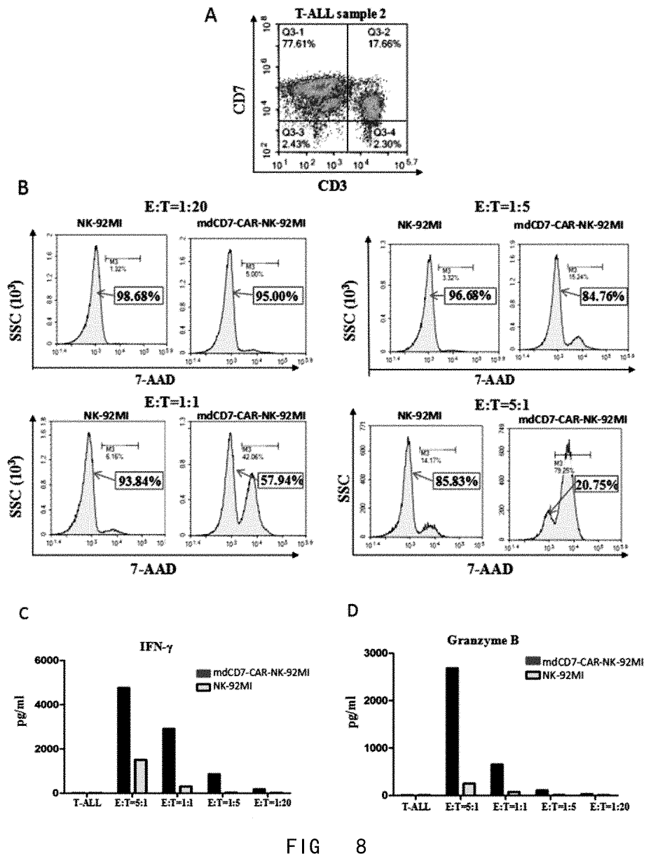

In order to further evaluate the cytotoxicity of mdCD7-CAR-NK-92MI cells to primary T-ALL tumor cells in vitro, the cytotoxicity of mdCD7-CAR-NK-92MI cells to primary T-ALL tumor cells in vitro was detected, and the cytokines produced after co-culture with primary T-ALL tumor cells were obtained. CD7 was highly expressed in the primary T-ALL tumor cells used (FIG. 8A). After 24 hours of incubation with primary T-ALL cells, the percentage of remaining tumor cells in the mdCD7-CAR-NK-92MI group was observed to be significantly lower than that in the control NK-92MI group at an effector-target ratio of 1:1 or 5:1 (FIG. 8B). The results show that mdCD7-CAR-NK-92MI cells have significant cytotoxicity to primary T-ALL tumors after 24 hours of co-culture. It was also observed that mdCD7-CAR-NK-92MI cells could produce high levels of IFN-γ and granzyme B (FIGS. 8C and 8D). These results show that mdCD7-CAR-NK-92MI has significant cytotoxicity to T-ALL tumor cells in vitro.

### Example 7 mdCD7-CAR-NK-92MI Cells have Significant Anti-Leukemia Activity In Vivo

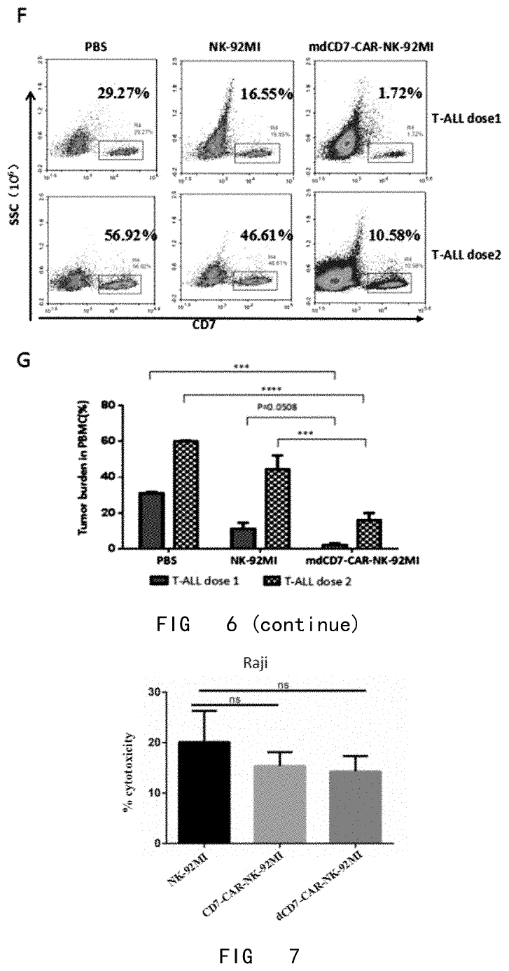

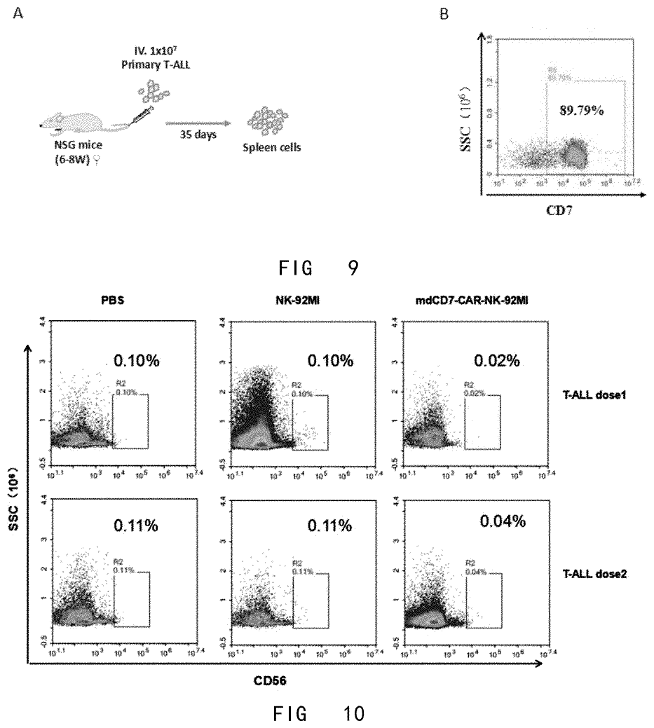

In order to evaluate the antitumor activity of mdCD7-CAR-NK-92MI cells in vivo, a mouse xenograftmodel was constructed using patient-derived T-ALL cells. Three B-NSG mice were injected with primary T-ALL tumor cells (1×10⁷ cells per mouse) via tail vein and the dying mice were euthanized (FIG. 9A). Spleen cells were ground, then treated with erythrocyte lysate, and detected by cell flow cytometry. There were 90% T-ALL tumor cells (FIG. 9B). The collected T-ALL cells were transplanted into 30 mice. The experimental scheme of animal experiment is shown in FIG. 6A. They were divided into two groups with 15 mice in each group. Each mouse in one group was injected with 2.0×10⁶ T-ALL cells (T-ALL dose 1), and mice in the other group were injected with 1.0×10⁷ T-ALL cells (T-ALL dose 2). After 3 days, NK-92MI or md-CD7-CAR-NK-92MI cells were administered every 2-4 days for a total of 5 times. Three days after the last administration, the tumor burden in peripheral blood of mice was measured by taking blood from eyelids. The results show that mdCD7-CAR-NK-92MI cells can significantly prolong the survival of mice compared with PBS control group and NK-92MI group (FIGS. 6C and E). The result of flow cytometry also demonstrates that mdCD7-CAR-NK-92MI inhibits T-ALL tumor cell proliferation in the xenogeneic mouse model (FIGS. 6F and G). The ratio of NK-92MI/CD7-CAR-NK-92MI cells in peripheral blood of mice was also measured by flow cytometry. The results showed that no NK-92MI/CD7-CAR-NK-92MI cells were detected in mice 3 days after the last administration (FIG. 10). It demonstrates that the duration of NK-92MI cells in mice is very short. The safety of NK-92MI cells is very good. In addition, for most mice, only slight weight loss was observed briefly (FIGS. 6B, D).

In conclusion, mdCD7-CAR-NK-92MI cells can significantly reduce tumor burden, control tumor growth and significantly prolong the survival time of B-NSG mice in PDX mouse model compared with NK-92MI control group.

### Example 8

In this example, the in vitro cytotoxicity of 8 monovalent CD7-CAR-NK-92MI and 5 bivalent dCD7-CAR-NK-92MI monoclonal cells to primary T-ALL tumor cells and significant anti-leukemia activity in vivo were also studied. The experimental method is the same as that of Examples 6 and 7, in which mdCD7-CAR-NK-92MI cells were replaced by 8 monovalent CD7-CAR-NK-92MI and 5 bivalent dCD7-CAR-NK-92MI monoclonal cells.

The results show that 8 monovalent CD7-CAR-NK-92MI and 5 other bivalent dCD7-CAR-NK-92MI also have significant cytotoxicity to T-ALL tumor cells in vitro, which is slightly lower than that of mdCD7-CAR-NK-92MI cells. At the same time, it is also found that 8 monovalent CD7-CAR-NK-92MI and 5 other bivalent dCD7-CAR-NK-92MI can also significantly reduce tumor burden, control tumor growth, and significantly extend the survival period of B-NSG mice.

### Discussion

CAR-T cells can achieve lasting remission for patients with refractory B-cell leukemia and lymphoma, but there is a lack of effective treatments for patients with T-cell malignancies. After extensive screening, CD7 is finally selected as a therapeutic target for T cell malignancies and AML. CD7 is highly expressed in most T cell malignancies and is not expressed in about 9% of normal peripheral T cells. In addition to T cell malignancies, CD7 is expressed in approximately 24% of AML cases and is considered to be a marker of leukemia stem cells. In addition, it is an antigen that is missing in T cells without affecting immune function. The CD7-deficient mouse model shows a normal lymphocyte population and maintains normal T cell function.

Although CD7 is an attractive and ideal target for T cell malignancies, effector T cells modified with CD7-CAR cannot significantly down-regulate the expression of CD7, leading to cannibalism between CAR-T cells and affecting T cell expansion. The use of autologous T cells to prepare CD7-CAR-T also faces many challenges. First of all, patients with recurrent T-ALL are usually pretreated with T-cytotoxic drugs. Therefore, the number and function of T cells may be significantly affected, affecting the preparation of active CD7-CAR-T cells. Secondly, most T cell hematological malignancies and normal T cell effectors express CD7 antigen, making it difficult to purify normal T cells from malignant T cells for CAR-T cell preparation. Therefore, this potential contamination risk limits the use of patient-derived T cells to prepare CAR-T cells to treat T cell malignancies.

In the present invention, monovalent CD7-CAR-NK-92MI and bivalent dCD7-CAR-NK-92MI cells were constructed based on the CD7 nanobody VHH6 sequence. Nanobodies have the advantages of small molecular weight, fast tissue penetration, high solubility and stability, high antigen binding specificity, and weak immunogenicity. Nanobody is an antibody fragment composed of a single monomer variable antibody domain derived from camelidae heavy chain antibody. Compared with the traditional scFv, the molecular weight of nano-antibody is smaller, so the CAR vector constructed with nanobody sequence is smaller and easier to transfect T cells. Moreover, nanobodies may produce lower immunogenicity than mouse antibodies. Specifically, due to the high sequence homology between the human VH framework and the nanobody framework and the short half-life, the nanobody can be rapidly cleared from the blood.

The present invention finds that CD7-CAR-NK-92MI and dCD7-CAR-NK-92MI cells have specific anti-tumor effects in vitro. It can specifically lyse CCRF-CEM and Jurkat cells in vitro. In addition, these cells also have specific anti-tumor effects on primary T-ALL tumor cells. 8 monovalent CD7-CAR-NK-92MI and 6 bivalent dCD7-CAR-NK-92MI monoclonal cell lines were screened in the present invention, and the activity of the monoclonal cells was compared. The mdCD7-CAR-NK-92MI monoclonal cell line is the most active cells of all monoclonal cell lines. In addition, in mouse experiments, mdCD7-CAR-NK-92MI cells can strongly reduce tumor burden, control tumor growth, and significantly extend the survival of primary T-ALL tumor model mice.

CAR-NK-92-based therapy can be used as a means to quickly clear tumor burden and bridge bone marrow transplantation. Different from CAR-modified autologous T cells, CAR-modified NK-92 or NK-92MI cells have the following advantages: (1) they can directly kill tumor cells by releasing granzymes, (2) due to their persistence in the body in a shorter time, fewer cytokines may be released, reducing the risk of cytokine storms, and (3) they can be developed into “off-the-shelf” products. The potential disadvantages of using NK-92 or NK-92MI cells in CAR treatment include lack of durability, and the curative effect may not be as good as CAR-T, but this can be overcome by repeated infusion of CAR-NK92 cells.

In a word, after a large number of research and screening, the CD7-CAR-NK-92MI constructed based on the anti-CD7 nanobody sequence for the first time shows certain therapeutic potential for T-ALL. The present invention not only confirms the specific cytotoxicity of CD7-CAR transduced NK-92MI cells to T-ALL in vitro, but also shows that CD7-CAR-NK-92MI has obvious inhibitory effect on tumor cells in PDX mouse model, and significantly prolongs the survival time of mice. The CD7-CAR-NK-92-MI cell of the present invention can be used as an independent treatment method or as a bridge connecting bone marrow transplantation.

All publications mentioned herein are incorporated by reference as if each individual document was cited as a reference in the present application. It should be understood that, after reading the above teachings of the present invention, those skilled in the art can make various modifications and changes. These equivalent forms are also within the scope defined by the claims appended hereto.

1. An engineered NK cell, which expresses a chimeric antigen receptor CAR, and the antigen binding domain of the CAR contains a VHH sequence of nanobody targeting CD7.

2. The NK cell of claim 1, wherein the structure of the antigen binding domain is V_{HH} or V_{HH}-I-V_{HH}, wherein the V_{HH} is the VHH sequence of nanobody targeting CD7, and I is none or a linking peptide La.

3. The NK cell of claim 1, wherein the antigen binding domain comprises a sequence as shown in SEQ ID NO.: 1 or 2.

4. The NK cell of claim 1, wherein the CAR has an amino acid sequence as shown in SEQ ID NO.: 3 or 4.

5. A chimeric antigen receptor CAR, wherein the antigen binding domain of the CAR contains a VHH sequence of nanobody targeting CD7.

6. A nucleic acid molecule encoding the chimeric antigen receptor CAR of claim 5.

7. A vector, wherein the vector comprises the nucleic acid molecule of claim 6.

8. A formulation, wherein the formulation comprises the engineered NK cell of claim 1 and a pharmaceutically acceptable carrier, diluent or excipient.

9. A method of prevention and/or treatment of cancers or tumors, which comprises: administering the engineered NK cell of claim 1 to a subject in need of.

10. A method of preparing the engineered NK cell of claim 1, wherein the method comprises the steps of introducing the nucleic acid molecule of claim 6 into the NK cell to obtain the engineered NK cell.
